(19) United States
(12) Patent Application Publication
(10) Pub. No.: US 2014/0100645 A1
(43) Pub. Date: Apr. 10, 2014

(54) MEDICAL DEVICE DELIVERY SYSTEM WITH AN INNER CATHETER HAVING A FLUSHING GROOVE

(71) Applicant: COOK MEDICAL TECHNOLOGIES LLC, Bloomington, IN (US)

(72) Inventors: Brent A. Mayle, Spencer, IN (US); James C. Merk, Terre Haute, IN (US)

(73) Assignee: COOK MEDICAL TECHNOLOGIES LLC, Bloomington, IN (US)

(21) Appl. No.: 14/045,296

(22) Filed: Oct. 3, 2013

Related U.S. Application Data
(60) Provisional application No. 61/709,472, filed on Oct. 4, 2012.

(51) Int. Cl. A61F 2/962; A61M 25/00

(52) U.S. Cl. CPC A61F 2/962; A61M 25/0021
USPC 623/1.11; 264/296

(57) ABSTRACT

A delivery system is provided with a flushing groove between an inner catheter and an outer sheath. The outer sheath is attached to a first handle member, and the inner catheter is attached to a second handle member. A medical device disposed between the inner catheter and outer sheath is deployed by moving the first and second handle members longitudinally relative to each other. The groove is provided along the outer surface of the inner catheter and is adapted to allow flushing fluid to pass between the inner catheter and the outer sheath.

This application claims priority to U.S. Provisional Application No. 61/709,472, filed Oct. 4, 2012, which is hereby incorporated by reference herein.

### BACKGROUND

The present invention relates generally to medical devices and more particularly to delivery systems for medical devices.

Intraluminal medical devices are used by physicians to treat numerous conditions using minimally invasive procedures. Examples of intraluminal medical devices include stents, stent-grafts, filters, valves, etc. One type of intraluminal medical device that has become especially common is self-expanding stents. Typically, self-expanding medical devices, including stents, are made from an elastic structure that may be compressed into a low profile state that can be passed through vessels in a patient with minimal trauma. Once at the desired treatment site, the self-expanding medical device is released and self-expands like a spring until it contacts a tissue wall which prevents further expansion. Common materials that are used in self-expanding medical devices include nitinol and stainless steel, although other materials are also possible.

Self-expanding stents are used to treat various organs, such as the vascular system, colon, biliary tract, urinary tract, esophagus, trachea and the like. For example, stents are commonly used to treat blockages, occlusions, narrowing ailments and other similar problems that restrict flow through a passageway. One area where stents are commonly used for treatment involves implanting an endovascular stent into the vascular system in order to improve or maintain blood flow through narrowed arteries. However, stents are also used in other treatments as well, such as the treatment of aneurysms. Stents have been shown to be useful in treating various vessels throughout the vascular system, including both coronary vessels and peripheral vessels (e.g., carotid, brachial, renal, iliac and femoral). In addition, stents have been used in other body vessels as well, such as the digestive tract.

One type of delivery system for intraluminal medical devices includes an inner catheter and an outer sheath attached to a handle arrangement. One portion of the handle is typically connected to the inner catheter and another portion of the handle is typically connected to the outer sheath. The inner catheter extends coaxially through the outer sheath, and the two portions of the handle are arranged to longitudinally pull the outer sheath relative to the inner catheter. Thus, when the distal end of the delivery system is positioned within the patient's body at the intended treatment site, the physician actuates the handle outside the patient's body by moving the two portions relative to each other so that the outer sheath is withdrawn over the medical device and inner catheter. In the case of self-expanding medical devices, like stents, the outer sheath also serves to radially restrain the device in the compressed state until the outer sheath is withdrawn. As the outer sheath is withdrawn, the medical device is released in the body at the treatment site, and in the case of a self-expanding stent, the stent expands outward away from the inner catheter and presses against the vessel wall. The handle may then be pulled by the physician to withdraw the inner catheter and outer sheath from the patient's body, while leaving the medical device implanted in the body.

Precise placement of intraluminal medical devices is a concern in most medical procedures. One problem that can contribute to imprecise placement of intraluminal medical devices is contraction and buckling of the inner catheter during deployment. This can be a particular problem in the deployment of self-expanding medical devices, like stents, because the medical device presses outward against the inner surface of the outer sheath prior to deployment. When the outer sheath is withdrawn, the outward pressure exerted by the medical device creates friction between the medical device and the outer sheath. Since the medical device is typically prevented from moving proximally with the outer sheath by a stop attached to the inner catheter, the frictional force between the medical device and the outer sheath causes the outer sheath to be in tension and the inner catheter to be in compression. This can cause the inner catheter to contract in length due to the compressive force. In addition, the inner catheter can buckle, or snake, within the outer sheath. Both of these responses can cause the distal end of the inner catheter, and thus the medical device itself, to move proximally from the intended treatment site. Although the contraction and buckling may decrease somewhat as the outer sheath begins to withdraw from the medical device due to the release of some of the frictional force, the distal end of the inner catheter may not completely return to the intended treatment site when the medical device is initially released and implants within the patient's body. Moreover, the stent and/or inner catheter can build up sufficient spring force due to the contraction of the inner catheter and the stent to cause the stent to jump distally once the static friction is released. With medical devices that cause high frictional loads against the outer sheath, like drug coated stents, covered stents and particularly long stents, the initial proximal movement of the inner catheter due to contraction and buckling and the subsequent distal movement due to the release of friction can make it difficult for a physician to predict the exact location where the medical device will be released in the patient's body.

Accordingly, the inventor believes it would be desirable to provide an improved delivery system for intraluminal medical devices.

### SUMMARY

An improved delivery system is described. The delivery system includes an outer sheath with an inner catheter disposed coaxially within the outer sheath. The inner catheter is provided with a groove along the outer surface of the inner catheter to allow flushing fluid to pass between the inner catheter and outer sheath. The inventions herein may also include any other aspect described below in the written description, the claims, or in the attached drawings and any combination thereof.

### BRIEF DESCRIPTION OF SEVERAL VIEWS OF THE DRAWINGS

The invention may be more fully understood by reading the following description in conjunction with the drawings, in which:

FIG. 1 is a side view of a delivery system;

FIG. 2 is an enlarged side view of the delivery system, showing the distal end of the delivery system;

FIG. 3 is a longitudinal cross-sectional view of the distal end of the delivery system;

FIG. 4 is an axial cross-sectional view of the inner catheter and outer sheath;

FIG. 5 is a side view of a multi-wire reinforcement structure;

FIG. 6 is a partial cross-sectional view of a method of forming the groove;

FIG. 7 is a longitudinal cross-sectional view of the distal end of the delivery system, showing longitudinal grooves;

FIG. 8 is an axial cross-sectional view of the inner catheter and outer sheath, showing the longitudinal grooves; and

FIG. 9 is a partial cross-sectional view of a method of forming the longitudinal grooves.

### DETAILED DESCRIPTION

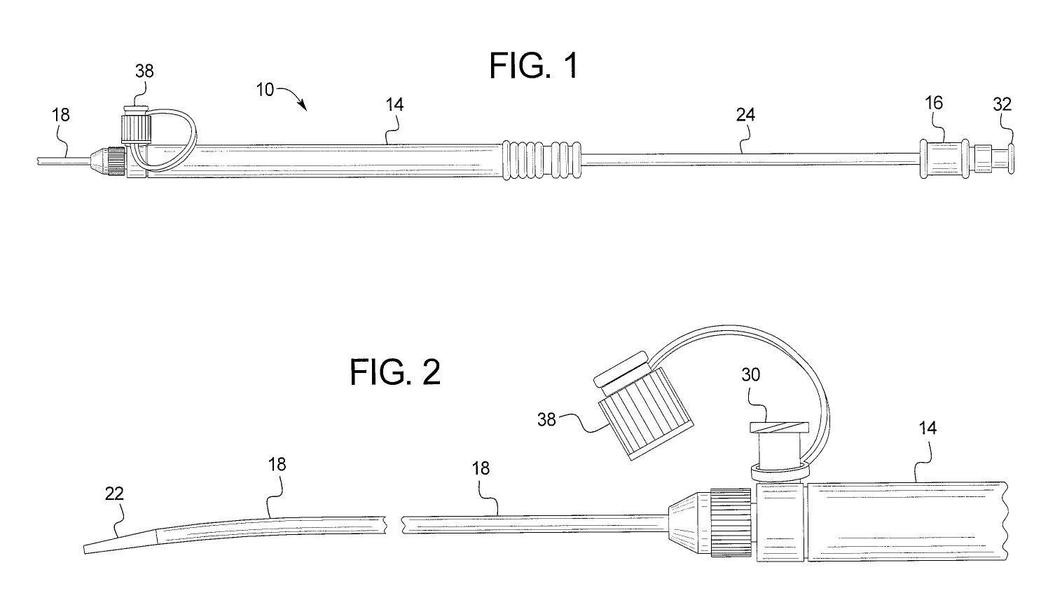

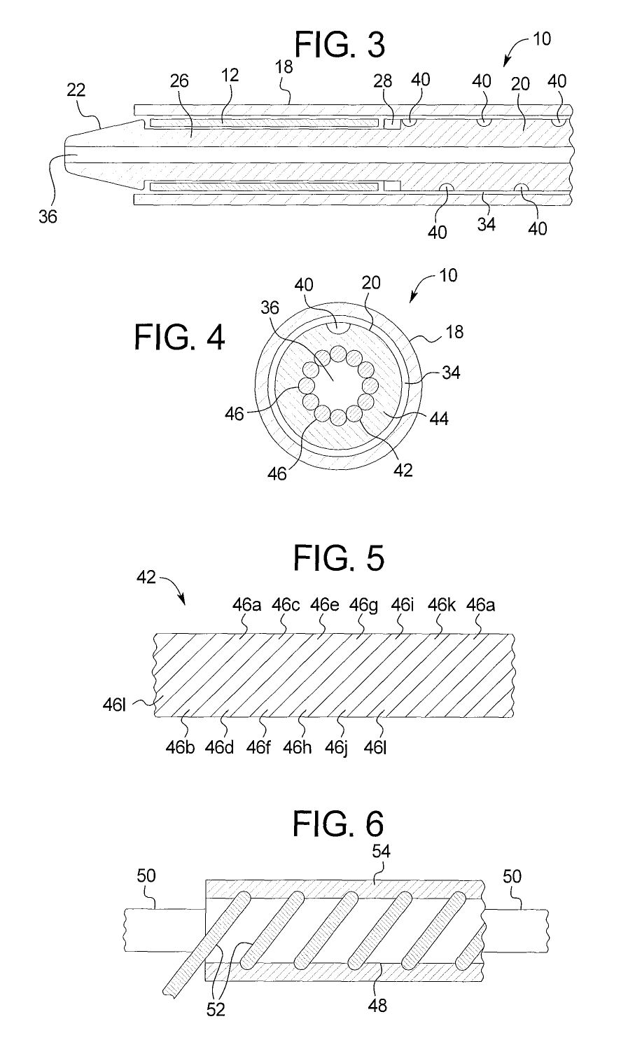

Referring now to the figures, and particularly to FIGS. 1-2, a delivery system 10 for a medical device 12 is shown. As shown in FIG. 1, the delivery system 10 includes first and second handle members 14, 16. The first handle member 14 is attached to an outer sheath 18, and the second handle member 16 is attached to an inner catheter 20 (shown in FIG. 3, and as tip 22 in FIG. 2). As shown in FIG. 1, the second handle 16 may be attached to a metal cannula 24 that extends through the first handle 14. The metal cannula 24 may be attached to the inner catheter 20 within the first handle 14. Although the first handle 14 is shown as a larger housing 14 and the second handle 16 is shown as a smaller knob 16, the design of the first and second handles 14, 16 could be reversed so that the second handle 16 is a larger housing and the first handle 14 is a knob that slides relative to the second handle 16.

As shown in FIG. 3, a medical device 12, such as a self-expanding stent 12, may be loaded into the distal end of the delivery system 10 between the inner catheter 20 and the outer sheath 18. The inner catheter 20 may be provided with a recessed area 26 to receive the medical device 12, and the outer sheath 18 may cover the outer region of the medical device 12. The inner catheter 20 may also be provided with a stop 28 adjacent the proximal end of the medical device 12. At the distal end of the medical device 12, the inner catheter 20 may be provided with a tapered tip 22 that extends past the distal end of the outer sheath 18 and is suitable for atraumatically passing through body passageways. The tip 22 of the inner catheter 20 may be integral with the inner catheter 20 or may be a separate component that is attached to the inner catheter 20 with an adhesive or the like.

The medical device 12 may be delivered into a cavity of a patient's body by positioning the distal end of the delivery system 10 in the patient's body at the desired treatment site, while the first and second handles 14, 16 remain outside the patient's body. Once the delivery system 10 is positioned so that the medical device 12 is located where it is intended to be implanted, the physician slides the first handle 14 relative to the second handle 16 while retaining the second handle 16 in a stationary position. This causes the outer sheath 18 to slide proximally relative to the inner catheter 20. Because the inner catheter 20 preferably does not move during the delivery step and the stop 28 on the inner catheter 20 prevents the medical device 12 from moving proximally with the outer sheath 18, the medical device 12 becomes uncovered and exposed as the outer sheath 18 moves proximally away from the medical device 12. In the case of a self-expanding medical device 12 like a stent 12, the stent 12 expands outward once it is released from the outer sheath 18 and expands until it contacts the wall of the body cavity.

As shown in FIG. 1, the delivery system 10 may be provided with first and second ports 30, 32. The first port 30 is in fluid communication with a space 34 between the inner catheter 20 and outer sheath 18, while the second port 32 is in fluid communication with a longitudinal lumen 36 extending through the inner catheter 20. As is conventionally understood, the second port 32 and inner catheter lumen 36 may be used with a guidewire passing therethrough to guide the delivery system 10 to the desired treatment site. The first port 30 is typically used to flush air out of the space 34 between the inner catheter 20 and the outer sheath 18, which includes the medical device 12 itself. Typically, a saline solution is used for flushing the system 10. The flushing fluid also serves as a lubricant between the inner catheter 20 and outer sheath 18 and between the medical device 12 and outer sheath 18. As shown in FIG. 2, the first port 30 may be provided with a cap 38 that threads onto the first port 30.

In conventional delivery systems, the outer diameter of the inner catheter 20 is typically sized at least 0.005″ smaller than the inner diameter of the outer sheath 18. This results in an annular gap 34 between the inner catheter 20 and the outer sheath 18 that has been deemed sufficient for flushing the space 34 between the inner catheter 20 and the outer sheath 18 and the medical device 12. However, one problem is that this annular gap 34 allows the inner catheter 20 to buckle, or snake, within the outer sheath 18 when a compressive load is applied to the inner catheter 20 during delivery of the medical device 12. Also, in order to maintain a conventional annular gap 34, the diameter of the inner catheter 20 must be reduced, which reduces the compressive stiffness of the inner catheter 20. In the improved delivery system 10 described herein, it is preferred that the annular gap 34 between the inner catheter 20 and the outer sheath 18 be minimized as much as possible to prevent the inner catheter 20 from buckling within the outer sheath 18 while still allowing the inner catheter 20 and outer sheath 18 to slide relative to each other. For example, it is preferred that the inner catheter 20 and outer sheath 18 be sized so that the clearance 34 between the nominal outer diameter of the inner catheter 20 and the nominal inner diameter of the outer sheath 18 be about 0.003″ or less. More preferably, the clearance 34 between the inner catheter 20 and the outer sheath 18 is about 0.0005″ to about 0.002″. As a result, with the tighter fit between the inner catheter 20 and the outer sheath 18, there is less open space around the inner catheter 20 that the inner catheter 20 can bend within when a compressive load is applied to the inner catheter 20.

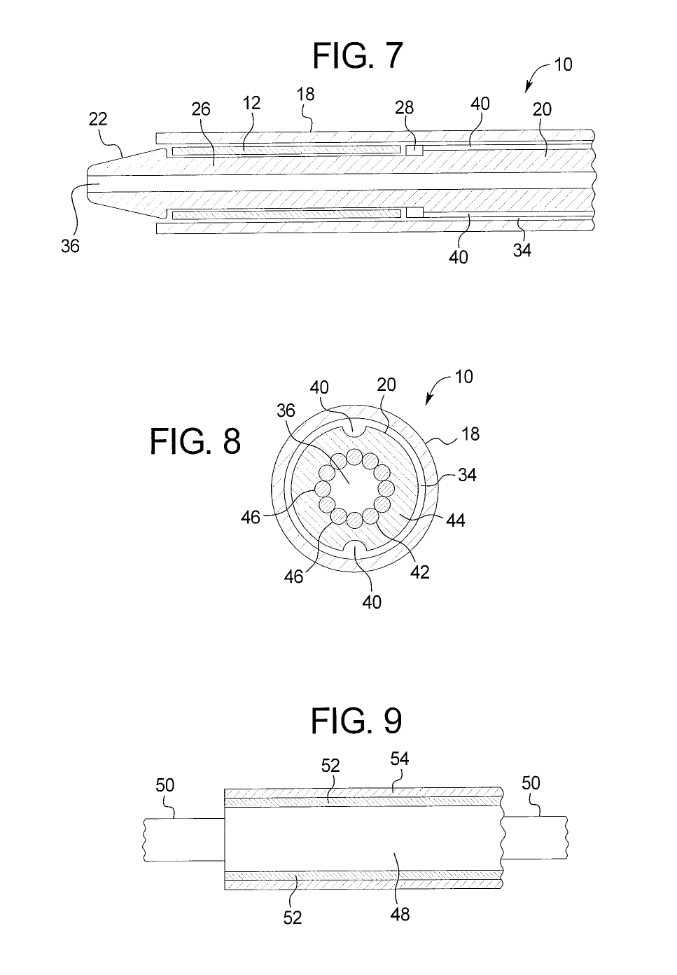

As shown in FIGS. 3-4, because of the smaller clearance between the inner catheter 20 and the outer sheath 18, the inner catheter 20 is provided with one or more grooves 40 along the outer surface of the inner catheter 20 to provide a passageway for flushing fluid to pass between the inner catheter 20 and the outer sheath 18. Although the groove 40 may extend along various lengths and portions of the inner catheter 20 and may extend helically or straight along the inner catheter 20, the groove 40 is preferably sized and positioned to pass flushing fluid through the groove 40 so that the flushing fluid is able to reach the medical device 12 and the distal end of the outer sheath 18. The groove 40 need not necessarily extend along the entire length of the inner catheter 20 since there may be portions of the inner catheter 20 and/or outer sheath 18 that are sized sufficiently to allow flushing fluid to flow therebetween without the groove 40. For example, portions of the inner catheter 20 may be tapered to a smaller outer diameter so that the clearance between the inner catheter 20 and outer sheath 18 is more conventional along the smaller outer diameter portion. However, it is preferred that the groove 40 extends along at least about 70% of the length of the inner catheter 20 between the proximal end of the medical device 12 and the proximal end of the outer sheath 18. More preferably, the groove 40 extends along substantially the entire length between the stop 28 on the inner catheter 20 and the first flushing port 30. Although, the groove 40 may extend helically around the inner catheter 20 as shown in FIGS. 3-4, it is also possible that the groove 40 may extend generally straight along the inner catheter 20 as shown in FIGS. 7-8. It is also possible that the inner catheter 20 may have multiple grooves 40, or the inner catheter 20 may have a single groove 40. For example, in the embodiment of FIGS. 3-4 where the groove 40 is helical, a single groove 40 may be used. However, in FIG. 7-8 where the groove 40 is longitudinal, multiple grooves 40 equally spaced around the circumference may be preferable. Alternatively, it may be possible for the groove 40 to be a web pattern along the inner catheter 20 instead of discrete, separated grooves 40.

Although it is possible that the groove 40 could have different cross-sectional shapes, it is preferred that the groove 40 have a round cross-sectional shape with a depth extending into the inner catheter 20 that is about half the diameter of the round cross-sectional shape or less. In order to allow sufficient flushing fluid through the groove 40, it is preferred that the depth of the groove 40 into the inner catheter 20 be at least about 0.001″ deep. It is also preferable for the depth of the groove 40 to be about 0.003″ or less.

In order to minimize compression of the inner catheter 20, it may also be desirable for the inner catheter 20 to have a reinforcement structure 42 below the groove 40. Typically, the groove 40 will be formed in a polymer portion of the inner catheter 20 that defines the outer surface of the inner catheter 20. Thus, as shown in FIG. 4, a polymer layer 44 may be disposed around a metallic reinforcement structure 42, and the groove 40 may be formed in the polymer outer layer 44. While it may be possible for the groove 40 to contact the reinforcement structure 42 at the bottom of the groove 40 (i.e., for the reinforcement structure 42 to be exposed through the groove 40), it is preferable for the groove 40 to be isolated from the reinforcement structure 42. This may be accomplished by controlling the depth of the groove 40 so that a portion of the polymer layer 44 remains disposed between the bottom of the groove 40 and the reinforcement structure 42. This may be useful to seal the flushing fluid passing along the groove 40 from the inner lumen 36 extending through the inner catheter 20. In other words, if the depth of the groove 40 extends down to the reinforcement structure 42 and the reinforcement structure 42 forms the inner lumen 36 without any other sealing structure, flushing fluid may be able to pass through the reinforcement structure 42 from the groove 40 to the inner lumen 36, or vice versa, from the inner lumen 36 to the annular gap 34. While this may be acceptable for certain products, it may be undesirable to allow communication between the groove 40 and the inner lumen 36.

Although different types of reinforcement structures 42 are possible, a particularly preferred reinforcement structure 42 is a solid tube 42 of a plurality of helically wound wires 46. As shown in FIGS. 4-5, the reinforcement structure 42 may have 12 wires 46a-I about 0.004″ in diameter that are positioned side-by-side so that there is substantially no gaps between each of the adjacent wires 46a-I. Thus, all of the wires 46 wind around the reinforcement structure 42 along the same helical angle. This provides a solid structure that is generally resistant to axial compression but is flexible to allow the inner catheter 20 to bend easily. However, as noted above, bending flexibility can allow an inner catheter 20 to buckle within the outer sheath 18 even without direct axial compression of the inner catheter 20. However, this problem may be overcome with the decreased annular clearance 34 between the inner catheter 20 and the outer sheath 18 described above. The decreased annular clearance 34 between the inner catheter 20 and outer sheath 18 also allows the inner catheter 20 to be built more robustly than otherwise possible in order to further resist compression and/or deflection of the inner catheter 20. For example, for a delivery system 10 with an outer sheath 18 having an outer diameter between about 0.071″ and about 0.083″, the cross-sectional diameter of each of the wires 46 in the reinforcement structure 42 may be about 0.008″ to about 0.010″.

FIG. 6 illustrates one method that may be used for making the inner catheter 20 with the groove 40 described above. A thermoplastic tube 48 which will form the inner catheter 20 may be disposed on a mandrel 50. The mandrel 50 may be approximately the size of the inner lumen 36 of the inner catheter 20. Preferably, the mandrel 50 is made of PTFE or has an outer layer of PTFE or other low friction coating to allow the mandrel 50 to be easily removed from the tube 48 after the groove 40 has been formed and other manufacturing steps have been completed. The thermoplastic tube 48 may be made from PEEK, urethane or nylon. As described above, the tube 48 may also have a reinforcement structure 42 if desired. One or more wires 52 may then be disposed on the outer surface of the thermoplastic tube 48. For example, a single wire 52 is helically wound around the tube 48. The wire 52 is also preferably coated with PTFE to allow easy removal after forming of the groove 40.

Heat shrink tubing 54, such as fluorinated ethylene propylene (FEP), may then be disposed over the wire 52 and the tube 48. Heat is then applied to the heat shrink tubing 54 and the thermoplastic tube 48 to cause the heat shrink tubing 54 to shrink in diameter and cause the thermoplastic tube 48 to soften. As a result, the heat shrink tubing 54 squeezes the wire 52 into the outer surface of the thermoplastic tube 48 to form one or more grooves 40 in the tube 48. The heat shrink tubing 54 and wire 52 may then be removed from the thermoplastic tube 48, with the thermoplastic tube 48 being left with a groove 40 formed in the outer surface thereof. Remaining steps, such as attaching the stop 28, forming or attaching the tip 22 and recessed area 26, and attaching the first and second handles 14, 16 may be done before or after forming the groove 40. As noted above, the mandrel 50 is preferably removed after the groove 40 is formed in the outer surface of the tube 48.

As shown in FIGS. 7-9, it may also be possible for the groove 40 to extend generally longitudinally along the outer surface of the inner catheter 20. In this arrangement, it is preferred that more than one groove 40 be provided, and that the grooves 40 be equally spaced circumferentially around the inner catheter 20. For example, the inner catheter 20 may be provided with two longitudinal grooves 40 oriented on opposite sides of the inner catheter 20 as shown in FIGS. 7-9. However, it may also be desirable to provide three equally spaced longitudinal grooves 40. Although more grooves 40 may not be necessary for flushing fluid, more grooves 40 could be provided if desired. A single longitudinal groove may also be used. As shown in FIG. 9, a similar method as described above may be used to form the longitudinal grooves 40. However, in order to form the longitudinal grooves 40, two or more wires 52 may be disposed longitudinally, as compared to helically, between the thermoplastic tube 48 and the heat shrink tubing 54.

Other methods may also be used to form the grooves 40 on the inner catheter 20. For example, a braided layer of wires, such as PTFE wires, may be applied to the outer surface of the inner catheter 20 in the manner described above. As a result, the grooves 48 may form a web pattern along the inner catheter 20. If the braided layer is tubular, it may be desirable to cut through the layer in order to remove it after the grooves 40 have been formed. Alternatively, the grooves 40 may be extruded onto the outer surface of the inner catheter 20. This may be particularly useful where the grooves 40 extend generally straight along the length of the inner catheter 20.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

We claim:

1. A delivery system for a medical device, comprising:
an outer sheath attached to a first handle member;
an inner catheter attached to a second handle member; and
a medical device disposed between said inner catheter and said outer sheath, said medical device being deliverable within a body cavity by moving said first handle member relative to said second handle member to slide said outer sheath proximally relative to said inner catheter, said outer sheath thereby exposing said medical device to said body cavity;
wherein said inner catheter comprises a groove disposed along an outer surface of said inner catheter and extending along a length of said inner catheter, said groove being adapted to pass flushing fluid therethrough between said inner catheter and said outer sheath.

2. The delivery system according to claim 1, wherein said medical device comprises a self-expanding device.

3. The delivery system according to claim 2, wherein said self-expanding device comprises a stent.

4. The delivery system according to claim 1, wherein said length is at least about 70% of a length between a proximal end of said medical device and a proximal end of said outer sheath.

5. The delivery system according to claim 4, wherein said length is substantially an entire length between a stop disposed proximally adjacent a proximal end of said medical device and a flushing port disposed on said first or second handle member.

6. The delivery system according to claim 1, wherein said groove extends helically around said inner catheter.

7. The delivery system according to claim 1, wherein a cross-section of said groove is round.

8. The delivery system according to claim 1, wherein a depth of said groove is at least about 0.001″.

9. The delivery system according to claim 8, wherein said depth of said groove is about 0.003″ or less.

10. The delivery system according to claim 9, wherein a cross-section of said groove is round.

11. The delivery system according to claim 1, wherein said inner catheter comprises a nominal outer diameter and said outer sheath comprises a nominal inner diameter, a clearance between said nominal outer diameter and said nominal inner diameter being about 0.003″ or less.

12. The delivery system according to claim 11, wherein said clearance is between about 0.0005″ to about 0.002″.

13. The delivery system according to claim 1, wherein said inner catheter comprises a reinforcement structure with a polymer outer layer disposed around said reinforcement structure, said groove being disposed in said polymer outer layer.

14. The delivery system according to claim 13, wherein a portion of said polymer outer layer is disposed between a bottom of said groove and said reinforcement structure, said portion sealing said flushing fluid from an inner lumen extending through said inner catheter and surrounded by said reinforcement structure.

15. The delivery system according to claim 13, wherein said reinforcement structure comprises one or more helical wires with substantially no gaps between adjacent wire windings.

16. The delivery system according to claim 15, wherein a cross-sectional diameter of said one or more wires is between about 0.071″ to about 0.083″ and an outer diameter of said outer sheath is about 0.008″ to about 0.010″.

17. The delivery system according to claim 1, wherein said medical device comprises a self-expanding device, said length is at least about 70% of a length between a proximal end of said medical device and a proximal end of said outer sheath, and said inner catheter comprises a nominal outer diameter and said outer sheath comprises a nominal inner diameter, a clearance between said nominal outer diameter and said nominal inner diameter being about 0.003″ or less.

18. The delivery system according to claim 17, wherein said groove extends helically around said inner catheter, and said inner catheter comprises a reinforcement structure with a polymer outer layer disposed around said reinforcement structure, said groove being disposed in said polymer outer layer.

19. The delivery system according to claim 18, wherein said self-expanding device comprises a stent, a depth of said groove is at least about 0.001″, said depth of said groove is about 0.003″ or less, a cross-section of said groove is round, said clearance is between about 0.0005″ to about 0.002″, and said reinforcement structure comprises one or more helical wires with substantially no gaps between adjacent wire windings.

20. The delivery system according to claim 1, wherein said inner catheter consists of a single said groove extending helically around said inner catheter.

21. The delivery system according to claim 1, wherein said inner catheter comprises at least two of said groove extending longitudinally along said inner catheter and being equally spaced around said inner catheter.

22. A method of making an inner catheter, said inner catheter adapted for use in a medical device delivery system comprising an outer sheath attached to a first handle member, an inner catheter attached to a second handle member, and a medical device disposed between said inner catheter and said outer sheath, said medical device being deliverable within a body cavity by moving said first handle member relative to said second handle member to slide said outer sheath proximally relative to said inner catheter, said outer sheath thereby exposing said medical device to said body cavity, wherein said inner catheter comprises a groove disposed along an outer surface of said inner catheter and extending along a length of said inner catheter, said groove being adapted to pass flushing fluid therethrough between said inner catheter and said outer sheath, said method comprising:
disposing a mandrel through a thermoplastic tube;
disposing a wire on an outer surface of said thermoplastic tube;
disposing a heat shrink tube over said wire and said thermoplastic tube;
heating said heat shrink tube and said thermoplastic tube, said thermoplastic tube thereby softening and said heat shrink tube squeezing said wire into said outer surface of said thermoplastic tube;
removing said heat shrink tube from said wire and said thermoplastic tube; and
removing said wire from said thermoplastic tube, a groove thereby being formed along said outer surface of said thermoplastic tube.
